(19) United States
(12) Patent Application Publication
(10) Pub. No.: US 2015/0080766 A1
(43) Pub. Date: Mar. 19, 2015

(54) Model And Apparatus For Predicting Brain Trauma From Applied Forces To The Head

(71) Applicant: The Trustees of Dartmouth College, Hanover, NH (US)

(72) Inventors: Songbai Ji, Lebanon, NH (US); Wei Zhao, Lebanon, NH (US)

(21) Appl. No.: 14/490,313

(22) Filed: Sep. 18, 2014

Related U.S. Application Data
(60) Provisional application No. 61/879,603, filed on Sep. 18, 2013.

(51) Int. Cl. A61B 5/11; A61B 5/00

(52) U.S. Cl. CPC A61B 5/11; A61B 5/7271
USPC 600/595

(57) ABSTRACT

A system for evaluating head injury uses instrumented helmets to transmit accelerometer readings to a computing system configured with machine readable code to determine angle and acceleration of impacts. The system has code to compare the angle and acceleration of the impact to thresholds and read at least one precomputed simulation result corresponding to entries near the impact in a database of precomputed head impact model simulations; and for displaying interpolations from the precomputed simulation result. In particular embodiments, the simulation result includes strain on neural tracts. A method of evaluating an impact includes transmitting accelerometer readings from instrumented helmets or any other sensors to the computing system upon impact to the sensors; determining at least angle and acceleration of the impact from the accelerometer readings; reading at least one precomputed simulation result corresponding to an entry in the database nearest in angle and acceleration to the impact; and displaying information from the simulation.

### RELATED APPLICATIONS

The present document claims priority to U.S. provisional patent application 61/879,603 filed Sep. 18, 2013, the disclosure of which is incorporated herein by reference.

### BACKGROUND

Much attention has been paid to brain injury in recent times. Many veterans of the conflicts in Iraq and Afghanistan have complained of mental problems that they blame on brain injury, such as concussions caused by proximity to explosions of various types of ordinance.

Sports-related brain injury is also an ongoing problem. Hockey, rugby, soccer, and football are not only popular, but are well known to pose risk of concussion when a ball is “headed,” when players collide or hit each other, whether by spearing or otherwise, or when players fall and strike their heads. The National Football League is facing litigation from nearly four thousand former players who allege lasting damage from injury from concussions suffered while playing football, and similar litigation may arise regarding college football. Helmet makers have also been sued by people who claim that helmets could be better designed. Even baseball and softball can result in brain injury, such as when players are hit in the head by thrown or batted balls, helmets are often worn by baseball players at bat. Bicyclists and motorcyclists are also subject to head injuries during accidents, often despite wearing helmets.

While some helmets, including football helmets, have been instrumented with sensors such as accelerometers so that forces applied can be measured, it is not always directly apparent from sensor data alone which players are injured, and to what extent they may be injured. Sensors that simply measure peak acceleration seem to lack specificity in predicting concussion.

It would be desirable to have improved ways to predict brain injury from various physical stimuli, to better determine when players should be removed from games and subjected to treatment, and to predict and measure the effect of ameliorative devices, such as helmets without having to test them on live people.

Brain injuries are also common in the elderly among the aging population. When the brain shrinks slightly due to age, or disease, it has more room to slosh within the braincase of skull; greater sloshing plus age-degraded blood vessels combine to produce a higher likelihood of hematoma in elderly when they suffer a blow to the head. This is exacerbated by the greater likelihood of falls in elderly people that may result in striking their head. It would be desirable to understand which people are at greatest risk, and to have effective protective devices usable by them.

It is known that, when an object strikes a human head, there may be effects on the brain both on the “coup” side, where the object struck, and on the opposite or “contra-coup” side; even if the skull remains intact and the brain is not penetrated, these effects can lead to bruising, swelling, confusion, even bleeding and, in some cases, death. The effects on both coup and contra-coup side of the head depend significantly on the dimensions, mechanical and physical properties of brain tissue and surrounding structures, including the skull, meninges, and cerebrospinal fluid, and how the brain is accelerated by the blow, and decelerated by the opposite side of the skull.

The human brain is not a mass of uniform density and composition. The brain contains fibrous white matter portions that have many directional, fibrous, nerve tracts, portions of “grey matter” with high numbers of neuron bodies, dendrites, and synapses, but fewer fibrous tracts, chambers that are filled with fluid. The brain surface is also highly folded, and is bathed in fluid contained in membranes, such as the dura. The brain and membranes are fed by a large number of blood vessels that are subject to rupture in some types of head injury, ruptured vessels can lead to accumulations of blood (hematomas) that can temporarily or permanently impair brain function, and which may require treatment such as surgical drainage. The fibrous tracts not only complicate modeling of the brain's mechanical response to blows, but strain on fiber tracts may in some cases cause neurological impairment, and potentially has a role in concussion and cumulative effects of multiple concussions over a player's career.

Computer modeling of brain has been proposed as a tool in helmet design, as disclosed in published patent application WO2012078730 entitled Model-Based Helmet Design To Reduce Concussions, the disclosure of which is incorporated herein by reference.

### SUMMARY

A system for evaluating head injury uses an instrumented helmet to transmit accelerometer readings to a computing system configured with machine readable code to determine angle and acceleration of an impact from the accelerometer readings. The computing system is configured with machine readable code to determine a suspicious impact by comparing the angle and acceleration of an impact to thresholds and to read at least one precomputed simulation result corresponding to an entry nearest in angle and acceleration to the impact in a database of precomputed head impact model simulation results resident in memory; and for displaying information derived from the at least one precomputed simulation result. In particular embodiments, the precomputed simulation result comprises strain on at least one neural tract.

A method of evaluating an impact includes transmitting accelerometer readings from an instrumented helmet to the computing system upon the helmet encountering an impact; determining at least angle and acceleration of the impact from the accelerometer readings; reading at least one precomputed simulation result corresponding an entry in the database nearest in at least angle and acceleration to the impact; and displaying information derived from the at least one precomputed simulation result.

### BRIEF DESCRIPTION OF THE FIGURES

FIG. 1 illustrates an apparatus for monitoring and analyzing blows to the head.

FIG. 2 is a flowchart of a method of using the apparatus of FIG. 1.

FIG. 3A is an illustration of angles of head hits to the head of a player who suffered a concussion, with hit intensity encoded in greyscale.

FIG. 3B is an illustration of angles of head hits to the head of a football offensive lineman who did not suffer a concussion, with hit intensity encoded in greyscale.

FIG. 3C is an illustration of density of precomputed DHIM results in the pcMRA database relative to impact angle for evaluating suspicious hits in football players.

FIG. 4. Is an illustration of a mesh model as used herein for simulation of mechanical properties of brain.

FIG. 5 is a table of parameters in the Dartmouth Head Impact Model (DHIM).

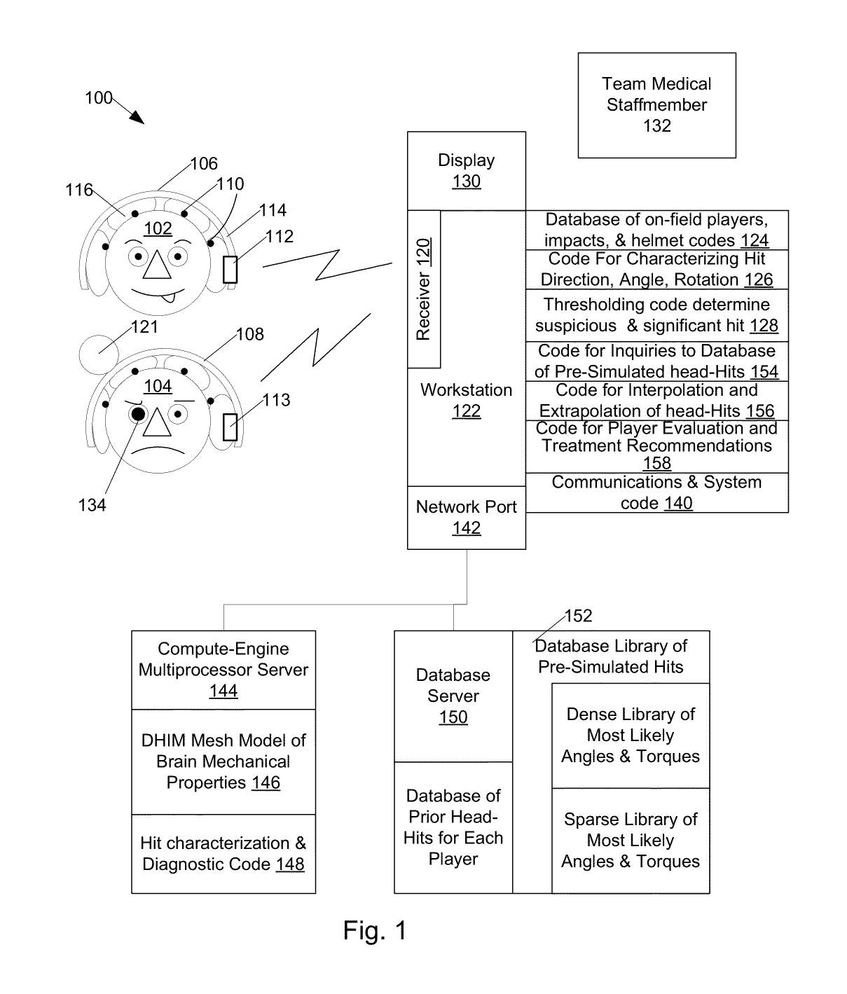

### DETAILED DESCRIPTION OF THE EMBODIMENTS

In an embodiment, a football or hockey game is played with a head-impact-analysis system 100 deployed. Each player 102, 104, of a team wears an instrumented helmet 106, 108, such as an instrumented football (Riddell Inc., Rosemont, Ill.) or hockey (Easton S9, Easton Sports, Scotts Valley, Calif.; CCM Vector, Reebok, Saint-Laurent, Quebec) helmet. In alternative embodiments, and for other sports or for army soldiers in combat conditions where head impacts are likely, other brands and styles of instrumented helmets may be used. For some other sports, such as professional boxing where helmets are typically not worn, a mouthpiece equipped with accelerometers may replace the instrumented helmet. Similarly, sensors may be embedded in a stick-on patch that may be stuck to a person's head. For purposes of this document, the term instrumented headgear includes any device attachable to a person's head that is configurable to measure accelerations undergone by that head, and thus to measure raw accelerations that may affect the person's brain.

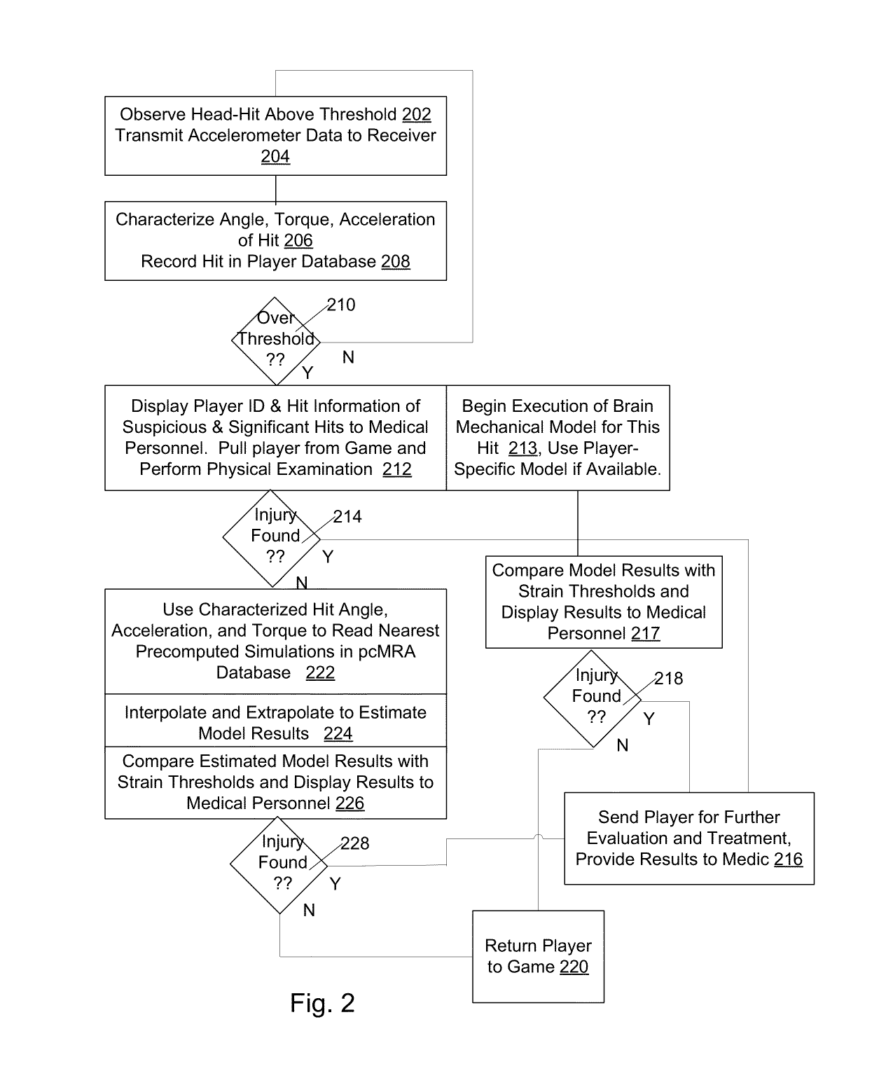

In the case of a helmet, each instrumented helmet has multiple accelerometers 110 positioned to be against a head of player 102, 104 when the helmet is worn, the sensors coupled to provide acceleration information to a digital radio transmitter 112, 113, together with a plastic shell 114 and sufficient padding 116 to prevent skull fractures as known in the helmet art. In a particular embodiment, the accelerometer and transmitters of helmets are the Head Impact Telemetry (HIT) System (Simbex, Lebanon N.H.) having six linear accelerometers located on a head-side of padding 116. Each transmitter has a unique identification code, transmitted with accelerometer readings, such that a receiver 120 in a workstation 122 can identify a particular helmet, and thus player, associated with each set of received accelerometer readings. Accelerometer readings are transmitted with a recent accelerometer reading history when any of the accelerometers of a helmet observe 202 (FIG. 2) a hit exceeding a pre-set threshold. In an exemplary embodiment, the pre-set threshold for transmitting accelerometer readings is 14.4 gravities (g), and when this threshold is exceeded a 40 millisecond (ms.) acceleration-time history data is transmitted by the transmitter and includes history from all accelerometers of that helmet.

Upon any impact by any object or player 121 to a helmet, and presumably a head of a player wearing the helmet, the transmitter, such as transmitter 113, in the affected helmet transmits 204 accelerometer readings to receiver 120 in workstation 122 together with its identification code. Workstation 122, typically located on sidelines at a sporting event, receives the accelerometer readings. The workstation then executes machine readable instructions 126 located in its memory to characterize 206 direction and angle, and peak magnitude, of linear and rotational (torque) accelerations associated with that impact. The workstation identifies a player's records associated with that transmitter and helmet in a database 124 located in its memory, the database including helmet identification codes and player identification (including player name) and player impact history information, and records 208 the characterized readings in database 124.

In the case of bicyclist, motorcyclist, and army combat helmets intended to be worn in the field or on open road, instead of a digital radio transmitter, the helmet is equipped with a recording device. The recording device is configured to record direction, angle, and peak magnitude of linear and rotational accelerations associated with events surpassing a threshold. Acceleration readings from such a helmet intended for field use are transmitted to the workstation by coupling the recording device to a workstation whenever it is desirable to analyze a possible head injury sustained by the wearer—such possible head injuries may result from accidents, impact of objects (such as bullets or shrapnel) on the helmet, or shockwaves from nearby explosions.

Once direction and acceleration, and rotational acceleration, associated with the impact are characterized, the workstation executes thresholding 210 code 128 to determine whether the impact is significant enough to be suspicious of possible head injury, warranting physical examination of a player and analysis in more detail. Code 128 has at least two thresholds, a first threshold for suspicious head-hits, and a second, higher, threshold for significant hits. If hits are below the suspicious threshold, the player is allowed to remain in the game. In a particular embodiment, the suspicious threshold is configurable for each individual player and each player's threshold is stored in the database.

Whenever an impact exceeds the suspicious threshold, player identification information is displayed 212, together with characterized acceleration information and a red flag if the significant-hit threshold is exceeded, on display 130 to a league or team medical official 132. Official 132 then calls the identified player 104 to sidelines and performs an on-field physical and mental status examination of the player. If 214 official 132 detects evidence of concussion, or finds other neurological impairment, such as disorientation, loss of consciousness, or a blown pupil 134, or if the higher significant hit threshold is exceeded, the official withdraws the player from the game and sends 216 the player to a medical facility for evaluation and treatment. Evaluation and treatment may include withdrawal from games for a time to allow healing, observation and neurological testing, computed x-ray tomography (CT) to locate intracranial bleeding with drainage if necessary, and other care.

The suspicious-hit threshold is set low enough that a significant percentage of head hits reaching this threshold are not above the significant-hit threshold, and are not associated with neurological impairment detectable by medical official 132 in a quick on-field examination; it is desired to evaluate players suffering these suspicious, but possibly not significant, hits in more detail before permitting the players to return to the game. It is also desirable to provide medical personnel with additional information useful in their evaluation of players who suffer hits exceeding the significant-hit threshold

We have developed a computational model of the human head, the Dartmouth Head Injury Model (DHIM) that includes mechanical modeling of anatomical regions of the brain as well as functionally important neural pathways. We have also developed a technique to derive white matter fiber strains (i.e., stretches along fiber orientations) in order to infer the risk of diffuse axonal injury based on thresholds determined from in vivo animal and in vitro brain injury studies. We believe our model includes functionally important neural pathways to assess their risk of injury that could relate to specific clinical symptoms, and we have evidence that white fiber strains correlate with concussive injuries. In addition, we propose to use white matter fiber strains instead of maximum principal strains that are commonly employed to assess the risk of concussion.

In an embodiment, while the characterized hit direction, angle, and rotation for a suspicious hit are being displayed to official 132, the hit direction, angle, and rotation are uploaded by communications and system code 140 through workstation network interface 142 to a compute engine server 144, typically a multiprocessor system, where the DHIM model 146 resides and, and the model is executed 213 to simulate hit-induced movement of brain tissue and resulting strain on fiber tracts within the brain; execution of the model is begun even before on-field physical examination in order to provide fast results. Upon completion of DHIM model 146 execution, server 144 executes hit characterization and diagnostic support code 148 to determine data regarding any neural tracts of the brain that may have suffered damage by comparing calculated strain on tracts to thresholds, including concussion-type damage, and to determine neurological signs that may be associated with damage to those tracts; determined over-strained tracts and associated neurological sign data are downloaded into database 124 and displayed 217 to medical staff 132 who then uses this data to further evaluate player 104 and makes a decision 218 whether player 104 is uninjured and may return 220 to the game or is injured and sent 216 for further evaluation and possible treatment. Model results, and associated neurological signs, are provided to treating medical personnel, and recorded in the precomputed head impact model response atlas 152 for later use should the same or another player suffer a similar hit.

Executing the mechanical model is time consuming, for our model requiring 50 minutes to simulate a 40 millisecond impact on an 8-processor machine at present, and other investigators models may require even more runtime; there is resistance to the idea of having possibly-uninjured, expensive, star quarterbacks or other top players, held out of games for entire quarters unnecessarily while awaiting computer simulation results. It is therefore desirable to provide an intermediate, estimated, evaluation while definitive DHIM model simulations continue executing.

We have also developed a pre-computational scheme that allows real-time, or near real-time, estimation of regional brain mechanical responses for on-field head impacts without significant loss of accuracy by using a pre-computed response atlas to assess the risk of concussion and serve as guideline for “return-to-play” for each single impact, as well as for determining cumulative effects of multiple repetitive head impacts for each athlete.

A database 152 or atlas of pre-simulated or pre-computed model responses (the pcMRA or pre-computed model response atlas) to suspicious head-hits is maintained on a database server 150. Suspicious hit characterized impact data is transferred to the database along with the athlete's unique identification by Code for Inquiries to Database of Pre-Simulated Head-Hits 154. The database has two separate atlases. One is for brain strain-related (including strain, stress, strain rate) responses as induced by head rotational accelerations. The other one is for brain pressure responses as induced by head linear accelerations. The strain-related response atlas is indexed by angle of rotational acceleration, rotational acceleration peak magnitude and duration. The pressure response atlas is indexed by angle of linear acceleration and linear acceleration magnitude. The precomputed results and responses corresponding to one or more entries nearest in angle, linear acceleration, and rotational acceleration to the suspicious characterized impact are read 222 from the pcMRA database; these results are interpolated and extrapolated 224 by interpolation and extrapolation code 156 from these nearest pcMRA entries to the acceleration angles and intensities of the present hit, thereby providing estimated model results.

Strain or pressure from the estimated model results are then compared 226 against thresholds to determine likelihood of concussive injury, and both model results and likelihood of injury are displayed 226 to medical personnel. If injury is found, the player is sent 228 off-field for further evaluation and treatment, if no injury is found the player may be returned 216 back into the game, and if a question remains the player may be held on the sidelines until full simulations 213 of the particular hit are completed. In an embodiment, code for player evaluation and treatment recommendations 158 is executed on the interpolated and extrapolated simulation results to advise medical staff member 132 of likelihood of injury and, by determining neural tracts likely to have suffered strain and comparing that strain with thresholds, and retrieving signs and symptoms that can be associated with those tracts from the database, determining particular signs and symptoms that may be expected in the player who suffered the hit if that player is in fact injured. The medical staff member may then conduct further examination of the player, in particular by looking for those signs and symptoms, before returning the player to the game.

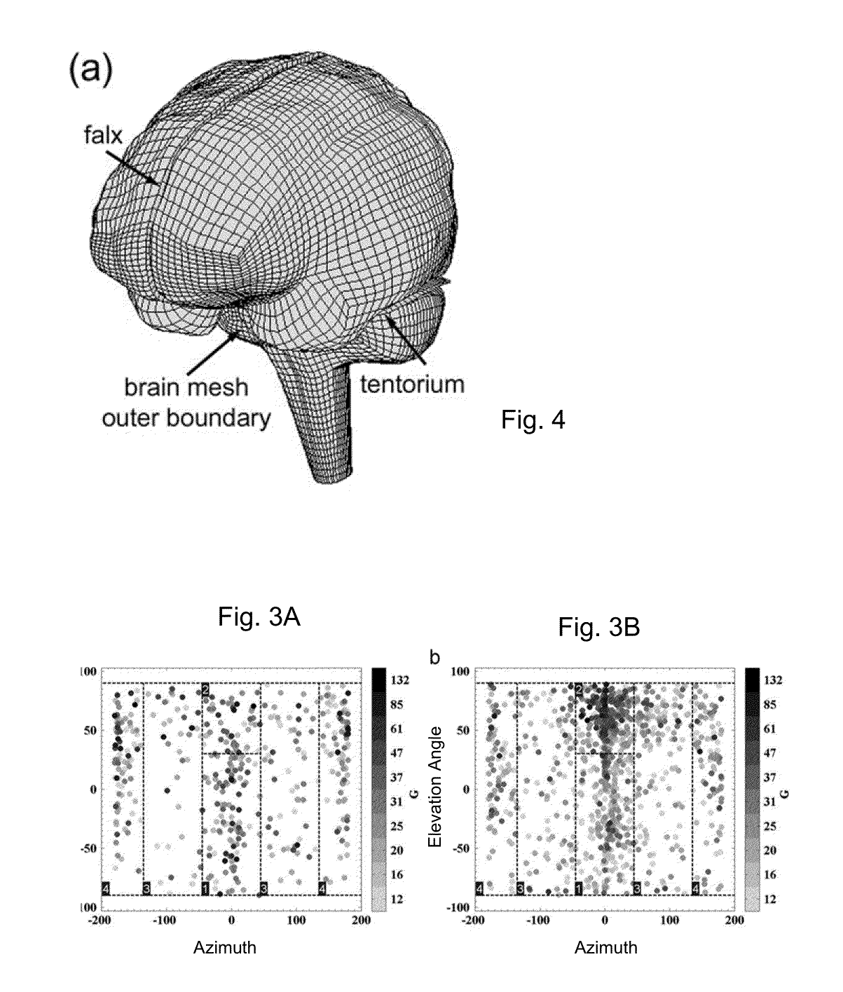

FIG. 3A illustrates hit angle in azimuth and elevation for head-hits suffered by a particular football player who suffered a concussion during two seasons of data-gathering. FIG. 3B illustrates hit angle in azimuth and elevation of head or helmet hits suffered by a football offensive lineman who did not suffer concussion during the same two seasons of play. As can be seen from FIGS. 3A and 3B, helmeted head hits are densest in angles aligned with the front and back of the helmet, not from the sides of the helmet, and linemen in particular have high densities of head hits from angles from thirty to ninety degrees above a horizontal axis and towards the helmet top. FIGS. 3A and 3B illustrate angle and linear acceleration, angular acceleration is not illustrated. It is expected that helmeted head hits in other sports may also have sport-specific predominant angles of impact.

The pcMRA atlas has a high or dense density of pre-simulated head impacts of suspicious and concussive intensity in a dense region 302 (FIG. 3C) of angles corresponding to those with high probability of forward impacts, a mid-dense region 304 corresponding to angles with medium probability of forward impacts, a mid-dense region 308 corresponding to angles with medium probability of rearward impacts, and a low density of pre-simulated head impacts in a region 306 of lateral angles corresponding to angles with a low probability of concussive impacts. The pcMRA has entries with a variety of angles of both linear and angular accelerations, and a variety of magnitudes of each of linear and angular accelerations.

Both linear and angular accelerations are important: linear acceleration mostly causes pressure gradients in the brain, while rotational acceleration causes strain (and stress) on particular nerve tracts. There is no consensus on which one (pressure vs. strain) causes brain injury. The pre-computed brain response atlas essentially builds a map of brain responses based on both linear and rotational accelerations. Note that these accelerations are vectors: not just magnitude, but also impact direction (angle) and an angle of rotation for rotational accelerations. While some prior work has considered only regional average responses (e.g., average strain in the left cerebrum), it is not just regional average of brain responses are important; their spatial distributions are also important, as that would indicate the location, and particular neural fiber tracts, where injury might occur.

Functions and neurological signs for many particular neural fiber tracts are known through other studies of head injuries and lesions; for example and not by limitation the optic nerves are tracts associated with vision, and some other particular tracts are known to be associated with movement, speech, hearing, and other known particular neurological functions, each of these functions may be associated with particular neurological signs and/or symptoms that medical personnel can look for, or test for. For example, tracts associated with Broca's area may be associated with speech disturbances, tracts extending from the optic nerve into occipital cortex with particular visual disturbances, and motor tracts leading from motor cortex to cerebellum, and from cerebellum to spinal cord, with movement disorders including ataxia. A map associating particular paths with particular neurological signs and/or symptoms that medical personnel may test for is stored in the pcMRA.

In an embodiment, scattered interpolation or grid-based interpolation is used to interpolate and extrapolate from the nearest pcMRA entries to determine stress on fiber tracts resulting from the present hit. In a particular embodiment, scattered interpolation or grid-based interpolation functions implemented in MATLAB are used, as illustrated in pseudocode below.
%% scattered interpolation sample code; input X is arbitrary, but is
% limited to 3D data at present
% first construct a scattered data interopolant, where X=[a, dt, loc],
% represents acceleration peak value, duration, and location (coded from 2D
% variable, theta and alpha angles), V is the mechanical response variable
% of interest, e.g., strain:
F=TriScatteredInterp(X, V);
% then for a given impact condition, Xp=[ap, dtp, locp] (“p” represents
% “point”, obtain the response by:
Vp=F(Xp);
%% grid-based interpolation sample code; input X has to be of a grid structure,
% but is not limited to 3D data
% suppose input conditions of 4D data, where “range” means the respective % range of data for each variable.
[a, dt, theta, alpha]=ndgrid(range_a, range_dt, range_theta, range_alpha];
% suppose the corresponding response is V, which has the same dimension of
% a, dt, theta, or alpha, then:

Vp=inerpn(a, dt, theta, alpha, V);

In an alternative embodiment, we locally define a linear regression model based on neighboring points and their associated response values and fit those points to a hyper-plane. Then the response values at the impact linear and rotational accelerations and directions of the present hit, or its location in the hyperplane, are found from the linear regression model.

### Interpolation and Extrapolation in a Particular Embodiment:

A testing dataset of 100 rotational impulses were created by randomly generating values for each individual variable within the corresponding range following a uniform distribution and then combining them with their randomly selected values. The ground-truth at each element was obtained via a direct simulation using each impulse as model input. By comparison, the pcMRA-interpolated was obtained through a multivariate linear interpolation operated independently for each element using values at neighboring 4D grid points in the atlas. Element-wise absolute differences in were obtained and further normalized by the ground-truth counterparts. Because the resulting normalized, element-wise differences constituted a spatial distribution within the FE domain; we reported the volume fractions above a range of percentage differences (varied from 0 to 100% at a step size of 1%) to characterize their response differences. Effectively, the reported volume fraction at each threshold level was analogous to an accumulated histogram.

The normalized differences relative to the ground-truths alone, however, did not necessarily reflect any clinical significance relative to injury-causing thresholds (e.g., the relative difference could be large in percentage but its magnitude may be sufficiently small and clinically irrelevant). Therefore, the element-wise response differences were further normalized by a range of injury thresholds (0.05-0.25 with a step size of 0.05) to evaluate the potential of deploying the pcMRA for real-world injury risk assessment. The range selected virtually encompassed thresholds established from an in vivo animal study (Lagrangian strain range 0.09-0.28 with an optimal threshold of 0.18, or equivalently, engineering strain range 0.086-0.249 with an optimal threshold of 0.166) and FE-based analyses of real-world injury cases (e.g., 0.19 in the grey matter,57 0.21 (0.26) in the corpus callosum (grey matter)0.28 Similarly, we investigated the volume fractions above a range of percentage differences for each injury threshold.

For each testing rotational impulse evaluated, we defined that the pcMRA-interpolated response was sufficiently accurate when the volume faction of large element-wise differences in (i.e. >10% relative to the ground-truth or a given injury threshold) for the whole-brain was less than 10% (dubbed the “double-10%” criterion). A success rate as the percentage of the testing impulses for which the pcMRA-interpolation was sufficiently accurate was used to evaluate the overall pcMRA interpolation accuracy.

Finally, because tissue-level regional responses can be conveniently used to assess region-specific risk of injury for a given ROI,28,47,57 we also computed the volume-weighted regional average for generic brain regions including the whole-brain, cerebrum, cerebellum and brainstem to evaluate the pcMRA estimation performance.

Similarly, the pcMRA estimate was considered sufficiently accurate when the absolute difference between the pcMRA-estimated response and the ground-truth was within 10% relative to the ground-truth or the same range of injury thresholds. Analogously, a success rate was used to assess the overall pcMRA performance in regional response estimate.

### Extrapolation

Because the head kinematic input variables of the training dataset were constrained within their ranges and did not encompass the entire sampling space, it was necessary to evaluate the pcMRA extrapolation performance. This was especially true because only a relatively small range of on-field measurements (50th-95th percentile values in on-field ice hockey) was covered. We did not evaluate the extrapolation performance for other variables because for the impulse duration, twice the standard deviation covered approximately 95.4% of occurrences (assuming a normal distribution). Although also restricted to a small range, they were intentionally limited in the feasibility study and could easily be expanded to cover the entire sampling space in the future.

To evaluate the extrapolation performance for below and above its range in the training dataset, two separate testing datasets (N=50 each) were randomly generated by constraining to a range either immediately below (500-1500 rad/s2) or above (4500-7500 rad/s2) that in the training dataset while maintaining the same ranges for other variables using the same approach described previously. The lower and upper end of eigenvalues for the below- and above-range extrapolation approximately corresponded to the 25th percentile subconcussive and the 95^{th} percentile concussive values for collegiate football, respectively. Element-wise whole-brain strain responses were obtained via a spline-based extrapolation using values at neighboring grid points in the pcMRA.

Similarly, we computed the volume fractions above a range of percentage differences (range 0-100%) in relative to the directly simulated ground-truth and the same range of injury thresholds, and further reported the success rates based on the “double-10%” criterion. In addition, the success rates for pcMRA-extrapolated regional strain responses for the same generic brain regions were also reported.

### Information Display and Medical Personnel

When information is displayed 212, 216, 226 to medical personnel, those medical personnel may use workstation 122 to review prior brain hit exposure of that individual player. Clinicians or coaches may utilize the simulation results along with the history of brain exposure (BE) for that individual player to assess the risk of injury, such as concussion, from repeated strains of the same tracts in short intervals ranging from days to weeks, and to lower rest or treatment thresholds when multiple hits causing strain on the same tracts create risk. The medical personnel thereby may make an informed decision as to whether a player may return to a game, or how long to rest before “return-to-play,” or whether off-field hospitalization or treatment is necessary. For data recorded by instrumented helmets worn in the field, the information may also be of use in determining appropriate medical care and likely rehabilitation needs of both injured wearers and of other people undergoing similar trauma.

Workstation 122, compute engine server 144, and database server 150 form components of a computing system. In alternative embodiments, the machine readable code for characterizing impacts, the pcMRA database, the code for reading precomputed model responses from the pcMRA and interpolating and extrapolating to determine strain and probable neurological symptoms, and the head injury model, are partitioned differently among machines of the computing system. For example, in an alternative embodiment, the pcMRA database resides not on a database server 150, but on workstation 122.

### The Dartmouth Head Injury Model (DHIM)

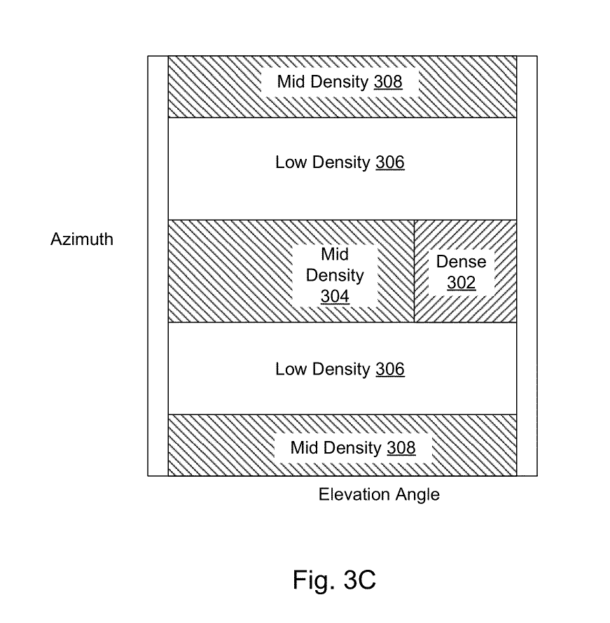

Our current head finite element (FE) model, the Dartmouth Head Injury Model (DHIM) was created based on a template high-resolution T1-weighted MRI (MRItemp) of an individual, the DHIM individual, selected from a group of concussed athletes. The model includes major intracranial components and simplified skull and scalp for the purpose of simulating brain responses relevant to sports-related concussion; the brain portion of the model is illustrated in FIG. 4. We incorporate anatomical regions derived directly from the neuroimaging atlas corresponding to the same individual to allow mechanical analysis of specific regions in the future. As a template for the model, we chose an individual whose head was normal in size and shape, and was representative of the athletic population. Template head position was neutral without tilting in the MR image space to align the anatomy-based coordinate system with that of the MRI. This alignment between the two coordinate systems enabled convenient transformation of FE model mesh nodes derived from MRI directly into the head anatomical intracranial physical space in order to properly apply biomechanical impact acceleration input, which is defined based on head anatomy.

To create the FE model, the imaged brain was first segmented from images stored in MRItemp. This generated a binary image volume from which an iso-surface was obtained to define the brain boundary surface geometry using an in-house MATLAB program. A completely automatic segmentation of the falx and tentorium is still not available at present (Penumetcha et al., 2011), so they were manually delineated on images from MRItemp. The resulting polygonal surfaces defining the anatomical geometries were then imported into Geomagic (Geomagic, Inc., Research Triangle Park, N.C., USA) for parameterization, and its results were imported into TrueGrid (version 2.3.4; XYZ Scientific Application, Inc., Livermore, Calif., USA) for meshing. Multi-blocks for the cerebrum, cerebellum, brainstem, as well as for the falx and tentorium were created based on the imported geometries, and “butterfly” topologies were used to project multi-block nodes onto the defining surfaces to ensure good mesh quality (Group-wise evaluation and comparison of white matter fiber strain and maximum principal strain in sports-related concussion. J. Neurotrauma. doi:10.1089/neu.2013.3268 (2014) by Songbai Ji, Wei Zhao, James C. Ford, Jonathan G. Beckwith, Richard P. Bolander, Thomas W. McAllister, Laura A. Flashman, Keith D. Paulsen, and Richard M. Greenwald). The outer surfaces of the projected blocks were then taken as a baseline surface to define elements for the cerebrospinal fluid (CSF), skull, and scalp through offsetting using Hypermesh (Altair Engineering, Inc., Troy, Mich.). Membrane structures of the pia and shell structures of the dura surrounding the CSF were also generated. To improve biofidelity in the basal region of the model, the segmented brainstem was extended to include part of the spinal cord along the neural axis (the spinal cord was not captured in MRI). An elastic membrane was also included at the base to simulate the loading environment for brainstem moving through the foramen magnum. Cortical bones and trabecular bones of the skull were represented by shell and solid elements with a thickness of 2 mm and ˜4 mm, respectively. The thickness of scalp was ˜5 mm. Because our head FE model was intended to study sports-related concussion for helmeted athletes where no skull fracture/deformation was observed or expected, the simplified representation of the skull/scalp and the omission of the face were acceptable because these structures deformation of these structures does not influence brain mechanical responses (both skull and scalp were represented by rigid bodies in on-field head impact simulations).

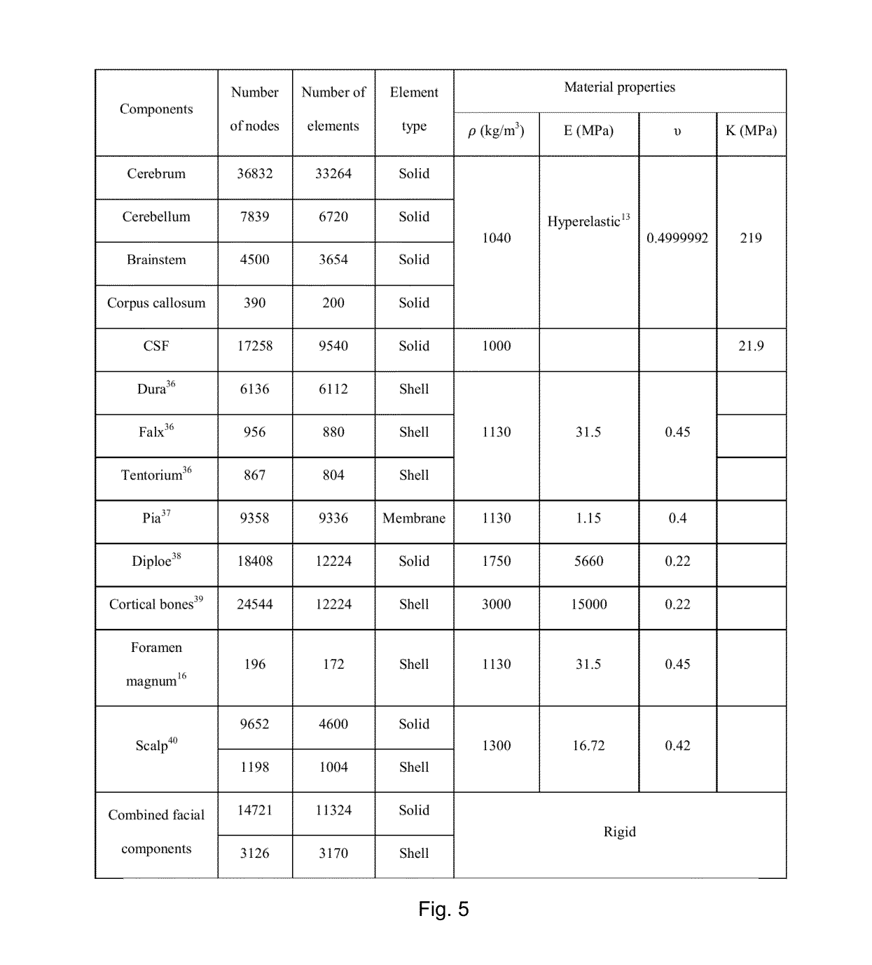

All solid parts (i.e., cerebrum, cerebellum, brainstem, CSF, skull, and scalp) were represented by hexahedral elements, while all surface parts (i.e., falx, tentorium, pia, dura, and the membrane at the base of the brainstem) were represented by quadrilateral elements. Reduced integration with hourglass control was used for all elements to ensure accurate simulation results (hourglass energy less than 8% of internal energy for a typical simulation). The CSF shared common nodes with adjacent parts including the brain, dura/skull, falx, and tentorium. A fluid-like property was used to simulate the CSF mechanical behavior. The details of the DHIM mesh components (number of nodes/elements) as well as the associated material model and property constants used in this study are summarized in FIG. 5 and Table 1. In total, the model contains 82952 nodes and 108965 elements with a combined mass of 4.349 kg. A summary of the mesh quality based on different criteria is listed in Table 2. All FE simulations were performed using Abaqus/Explicit (Version 6.12; Dassault Systèmes Simulia Corp., Providence, R.I.) in memory on a Linux cluster (Intel Xeon X5560, 2.80 GHz, 126 GB memory). The typical run time for a 40 ms. head impact was about 50 minutes with 8 CPUs.

TABLE 1
hyperelastic and viscoelastic material model showing Ogden constants
μᵢ and αᵢ and Prony constants gᵢ and τᵢ.

| μ₁ (Pa) | α₁ | μ₂ (Pa) | α₂ |
|---|---|---|---|
| 271.7 | 10.1 | 776.6 | −12.9 |

| | i = 1 | i = 2 | i = 3 | i = 4 | i = 5 | i = 6 |
|---|---|---|---|---|---|---|
| gᵢ | 7.69E−1 | 1.86E−1 | 1.48E−2 | 1.90E−2 | 2.56E−3 | 7.04E−3 |
| τᵢ (sec) | 1.0E−6 | 1.0E−5 | 1.0E−4 | 1.0E−3 | 1.0E−2 | 1.0E−1 |

TABLE 2

| | | Failure | |
|---|---|---|---|
| Parameter | Criterion | Percentage | Max/Min value |
| Warpage (°) | <35.20 | 1% (1%) | 116.43 (116.43) |
| Aspect | <10.70 | ~0% (~0%) | 11.95 (26.51) |
| Skew (°) | <64.00 | ~0% (1%) | 85.08 (85.08) |
| Min length (mm) | >0.70 | 0% (1%) | 0.742 (0.103) |
| Jacobian | >0.47 | ~0% (1%) | 0.24 (0.24) |
| Min angle (°) | >16.69 | ~0% (~0%) | 4.55 (4.55) |
| Max angle (°) | <160.65 | ~0% (1%) | 178.56 (178.56) |

### Computation of WM Fiber Strains

The εₑₚ and the strain tensor were extracted from the simulation results. To compute εₙ, fiber orientation at each WM (white matter) voxel was first obtained based on the primary eigenvector using ExploreDTI. The P-1 analysis was limited to the WM region by applying a binary image mask. FIG. 6 illustrates a typical axial image with color-coded WM fiber orientations. The WM voxels and their fiber orientation vectors were transformed into the global coordinates for analysis.

For each transformed voxel or sampling point originally in the DTI image space, a local coordinate system, xyz, was established with its origin identical to the transformed voxel location and the z-axis along the fiber orientation transformed from DTI image space into the coordinate system of the head finite element model. The x- and y-axis were arbitrarily established, as they did not influence the strain component of interest. A spatial transformation from the global to the local coordinate systems, T, was determined via singular value decomposition. For each sampling point, the strain tensor corresponding to its closest element (typical distance of 1.7+0.6 m relative to element centroid) was transformed to compute ε′ in the local coordinate system following tensor transformation. The WM fiber strain, or the stretch along the local z-axis, was readily obtained.

The peak strains at each sampling point were defined as their respective maximum values during the entire impact regardless of the time of occurrence. The WM volume fractions with large strain were compared above a number of representative thresholds for axonal damage drawn from an in vivo animal study that measured morphological injury and electrophysiological impairment. Five thresholds with four unique values (two were identical) were chosen that corresponded to the lower and upper bound (0.09 and 0.18) and the average (0.13) of a conservative threshold, and an optimal (0.18) and an average liberal (0.28) threshold during a particular study where we compared simulated strains to simulation results of concussed athletes diagnosed through other means. These values encompass thresholds established from other real-world injury analyses (e.g., 0.21 in the corpus callosum or 0.26 in the grey mater, or 0.19 in grey matter. Because regions exposed to high strains potentially indicate injury locations, it is important to compare the spatial distributions of regions with high strains determined by the two strain measures at each location to the threshold, for which their Dice coefficient readily serves the purpose.

### Computation of Pressure Responses

In addition to strain, pressure may be involved in brain injury. The system is therefore configured to simulate mechanisms of brain pressure in translational/direct impact. Because of the unique head shape where a larger curvature of the skull occurs in the forehead that results in a smaller brain-skull contact area in this location, the brain frontal region often sustains larger pressure for a given hit acceleration αₗᵢₙ irrespective of whether the impact is frontal or occipital. This finding suggests that the brain frontal region is likely more vulnerable to pressure-induced injury, which appears to agree well with many clinical observations. Further, because brain pressure is linearly proportional to αₗᵢₙ, only a baseline response along each given translational axis is necessary to directly determine P_{coup and P}_{c-coup} (pressures coup (on the side of brain facing the blow) and contra-coup (on the opposite side of brain)) without the need to recompute. Therefore, only two independent variables characterizing the directionality of the translational axis (the azimuth and elevation angles) are necessary to establish a pre-computed pressure response atlas subject to isolated αₗᵢₙ, or more realistically, αₗᵢₙ-dominated head impact, as opposed to four independent variables for the brain strain response atlas. Such a pre-computed atlas is essentially a profile of element-wise distribution of pressure values for each discrete translational axis to allow an instantaneous estimation of brain pressure responses at the tissue level (i.e., interpolated at every element throughout the brain) without a time-consuming direct simulation that typically requires hours or more on a high-end computer or even a super computer Although debate still exists whether αₗᵢₙ-induced brain pressures could also contribute to mild injuries such as sports-related concussion on the field because many believe αᵣₒₜ (rotational accelerations) as opposed to αₗᵢₙ causes diffuse axonal injury, at the minimum the pressure response atlas appears directly functional whenever it is desired to include pressure in head injury classification criteria. Together with the pre-computed brain strain response atlas, these tools have potential to increase throughput in head impact simulation, and therefore, allow exploration of the biomechanical mechanisms of traumatic brain injury in general as well as performing on-field screening of players to predetermined head injury criteria.

### Customized Head Models for Individual Athletes

There are some players who have very high value to a team, including quarterbacks. For many of these high-value players, an MRI of the head is already available or may be obtained at low cost for these players, instead of using the generic DHIM, a head impact model customized for that high-value player is prepared in the same manner as the generic DHIM was prepared from the DHIM individual.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. A system for evaluating head injury comprising:
an instrumented headgear configured to transmit accelerometer readings to a computing system;
the computing system configured with machine readable code to determine angle and acceleration of an impact from the accelerometer readings;
the computing system configured with machine readable code to determine a suspicious impact by comparing the angle and acceleration of the impact to thresholds;
a database of precomputed head impact model simulation results resident in a memory of the computing system;
the computing system configured with machine readable code adapted to read at least one precomputed simulation result corresponding an entry in the database nearest in at least angle and acceleration to the suspicious impact; and
the computing system configured with machine readable code adapted to display information derived from the at least one precomputed simulation result.

2. The system of claim 1 wherein the precomputed simulation result is derived by executing a finite element model derived from magnetic resonance images of a head.

3. The system of claim 1 wherein the computing system comprises a memory configured with a finite element model derived from magnetic resonance images of a head, the computing system configured to execute the finite element model on the angle and acceleration of the suspicious impact.

4. The system of claim 1 wherein the precomputed simulation result comprises strain on at least one neural tract.

5. The system of claim 4, further comprising database entries linking the at least one neural tract to a list of symptoms related to that neural tract, and further comprising machine readable code adapted to display the list of symptoms to medical personnel.

6. The system of claim 1 wherein the determined angle and acceleration of an impact includes a linear acceleration magnitude and associated angle, and a rotational acceleration magnitude and associated angle.

7. The system of claim 6 further comprising machine readable instructions adapted to interpolate between precomputed simulation results to determine strain on at least one neural tract associated with the suspicious impact.

8. The system of claim 7, further comprising database entries linking the at least one neural tract to a list of symptoms related to that neural tract, and further comprising machine readable code adapted to display the list of symptoms to medical personnel.

9. A method of evaluating an impact to a human head comprising:
transmitting accelerometer readings from an instrumented headgear worn by the human head to a computing system upon the helmet encountering an impact;
determining at least angle and acceleration of the impact from the accelerometer readings;
reading at least one precomputed simulation result from a database, the precomputed simulation result corresponding an entry in the database nearest in at least angle and acceleration to the impact; and
displaying information derived from the at least one precomputed simulation result.

10. The method of claim 9 wherein the precomputed simulation result is derived by executing a finite element model derived from magnetic resonance images of a head.

11. The method of claim 10 further comprising
sorting impacts into at least insignificant, suspicious, and serious impact categories based upon the accelerometer readings, and
executing a finite element model of a brain on the angle and acceleration of at least some suspicious impacts.

12. The method of claim 10 wherein the precomputed simulation result comprises strain on at least one neural tract, comparing strain to thresholds, and determining stressed neural tracts.

13. The method of claim 12 wherein the determined angle and acceleration of an impact includes a linear acceleration magnitude and associated angle, and a rotational acceleration magnitude and associated angle.

14. The method of claim 12 further comprising interpolating between precomputed simulation results to determine strain on at least one neural tract associated with the suspicious impact.

15. The method of claim 14, further comprising displaying a list of symptoms related to stressed neural tracts.

16. The method of claim 12, further comprising displaying a list of symptoms related to stressed neural tracts.

17. The method of claim 10 wherein the instrumented headgear is an instrumented football helmet, and wherein the information displayed comprises information indicating whether the impact is a possible concussive impact.

18. The method of claim 17 wherein the information displayed comprises information regarding possible symptoms associated with neural tracts stressed by the impact.
